(19) United States
(12) Patent Application Publication
(10) Pub. No.: US 2012/0283778 A1
(43) Pub. Date: Nov. 8, 2012

(54) DEVICE FOR CONNECTING TRANSVERSE BEAM AT TRIANGULAR POSITION OF VERTEBRAL LAMINA

(76) Inventor: Chung-Chun YEH, Taipei City (TW)

(21) Appl. No.: 13/553,828

(22) Filed: Jul. 20, 2012

Related U.S. Application Data
(63) Continuation-in-part of application No. 12/750,942, filed on Mar. 31, 2010.

(51) Int. Cl. A61B 17/70

(52) U.S. Cl. 606/250

(57) ABSTRACT

A fixing device for securing vertebral lamina includes a transverse rod, a vertical rod having a length longer than that of the transverse rod and a joint having a vertical hole to securely receive therein a portion of the vertical rod and a cutout communicating with the vertical rod to receive therein a portion of the transverse rod.

### CROSS-REFERENCE TO RELATED APPLICATIONS

This application is a continuation-in-part of a co-pending non-provisional application, titled “Method of Connecting Transverse Beam at Triangular Position of Vertebral Lamina”, with Ser. No. 12/750,942 and filed on Mar. 31, 2010 by the same applicant of this instant application. The description thereof is incorporated herein by reference.

### BACKGROUND OF THE INVENTION

1. Field of the Invention

The present invention relates to a device for connecting a transverse beam at a triangular position of a vertebral lamina, and more particularly to a device used for fixing a transverse beam at a triangular intersection position between a spinous process and a lamina of vertebral arch of a human spine for connecting and fixing a posterior spine.

2. Description of Related Art

Most conventional devices for connecting and fixing the posterior spine adopt a pedicle screw fixation system 1 (as shown in FIGS. 1a and 1b) having a plurality of pedicle screws 13 that are to be extended into pedicle 12 and fixed onto the vertebral bodies respectively. Each head of the pedicle screws is connected to a link rod 14 in the direction of a vertebral column for securely connecting the pedicle screws 13 to the vertebral column nearby to achieve the desired purpose.

However, even the conventional methods have been adopted for connecting and fixing a posterior spine for a long time, yet they still have drawbacks and may not be suitable for some situations. For instance, the pedicle screw fixation system 1 is not applicable for certain spinal diseases such as the collapse or osteoporosis of vertebral bodies, because the screw 13 section of a pedicle may not be able to be fixed onto the vertebral bodies effectively, and the pedicle screw fixation system 1 on the spine may become loose after a period of time, which may pose a threat to the patient and a surgery may be required to adjust the loosened screws.

It is to be noted that once the conventional pedicle screw fixation system 1 is used in a patient's vertebral column, the system 1 can no longer be associated with other connection devices. For example, any interspinous process fixation device cannot be connected directly and effectively to other fixation system(s).

Therefore, the aforementioned conventional pedicle screw fixation system and related surgical methods still have their drawbacks and inapplicability, and thus require further reviews, improvements and it is necessary to develop a device for connecting a transverse beam at a triangular position of a vertebral lamina to overcome the problems.

### SUMMARY OF THE INVENTION

The objective of the present invention is to provide a device for mounting a transverse beam at a triangular position of a vertebral lamina, which strengthens patients with unstable spinal structure the stability of the vertebral body via the device of the present invention.

In order to achieve the above purpose, the present invention includes a transverse rod providing a transverse fixation to a vertebral body; two joints respectively having an accommodating space for connecting two ends of the transverse rod and receiving one end of the vertical rod orthogonal to the transverse rod; and two screws for fastening the transverse rod, the vertical rod and the joints into one piece.

Those above mentioned elements construct a fixing element for fixation of the vertebral body. In order to fix a plurality of vertebrae, each joint is respectively fixed on each vertebra via the assistance of the transverse rod first, and connects with the other joints via the vertical rods, so as to tightly fasten the vertebrae in both vertical and horizontal orientations.

In addition, the fixing element has abilities for connecting to a varieties of other fixation elements, such as an inter-spinous process fixation device, a Pedicle screw or a spinal hook, so as to allow the device for connecting a transverse beam at a triangular position of a vertebral lamina to provide fixation function between each vertebral body with different fixation elements.

The invention, as well as its many advantages, may be further understood by the following detailed description and drawings in which:

### BRIEF DESCRIPTION OF THE DRAWINGS

FIGS. 1a and 1b are schematic views of a conventional pedicle screw fixation system;

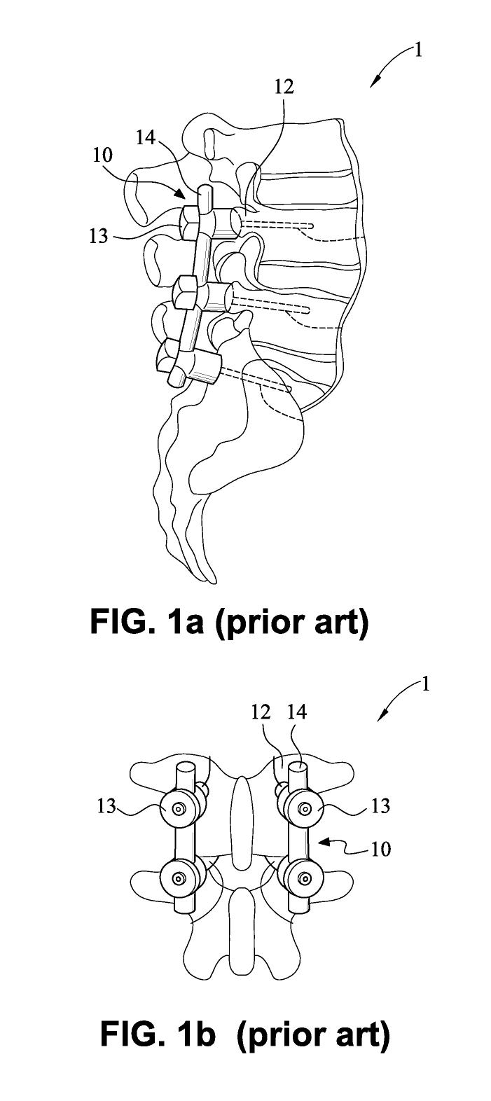

FIG. 2 is a flow chart explaining how the device constructed in accordance with the present invention is implemented;

FIG. 3a is a perspective view of a lamina of vertebral arch of a vertebra;

FIG. 3b is a top plan view showing a vertebra in FIG. 3a;

FIG. 3c is a perspective view showing the location of a transverse hole of a vertebra;

FIG. 4 is an exploded perspective view of a fixing element in accordance with the present invention;

FIGS. 5a and 5b are exploded perspective views showing the application of the fixing element in the vertebral lamina; and

FIG. 6 is a plan view showing the fixing elements connected to a linear rod element for connecting to a transverse beam.

### DETAILED DESCRIPTION OF THE INVENTION

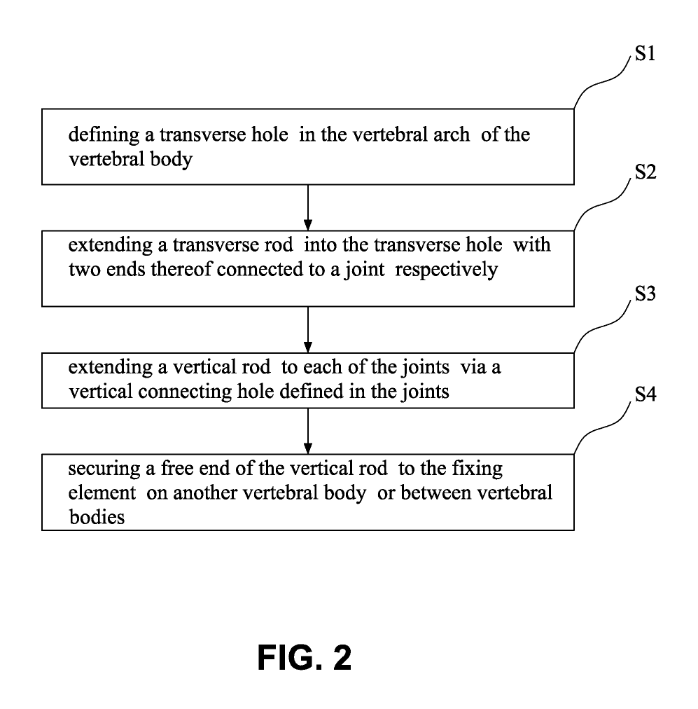

With reference to FIGS. 2 to 6, the device for connecting a transverse beam at a triangular position of a vertebral lamina constructed in accordance with the present invention includes at least one fixing element 2, which is used for fixing a vertebra. The connecting steps of the present invention are as the following: (shown as FIG. 2)

Step S1, defining a transverse hole 31 in the vertebral arch 302 of the vertebral body 30, wherein the transverse hole 31 is defined in an area between the bottom vertex of the lamina of vertebral arch and the top vertex of the spinous process;

Step S2, extending a transverse rod 21 into the transverse hole 31 with two ends thereof connected to a joint 24 respectively;

Step S3, extending a vertical rod 22 to each of the joints 24 via a vertical connecting hole defined in the joints; and

Step S4, securing a free end of the vertical rod 22 to the fixing element 2 on another vertebral body 30 or between vertebral bodies (not shown in FIG. 2).

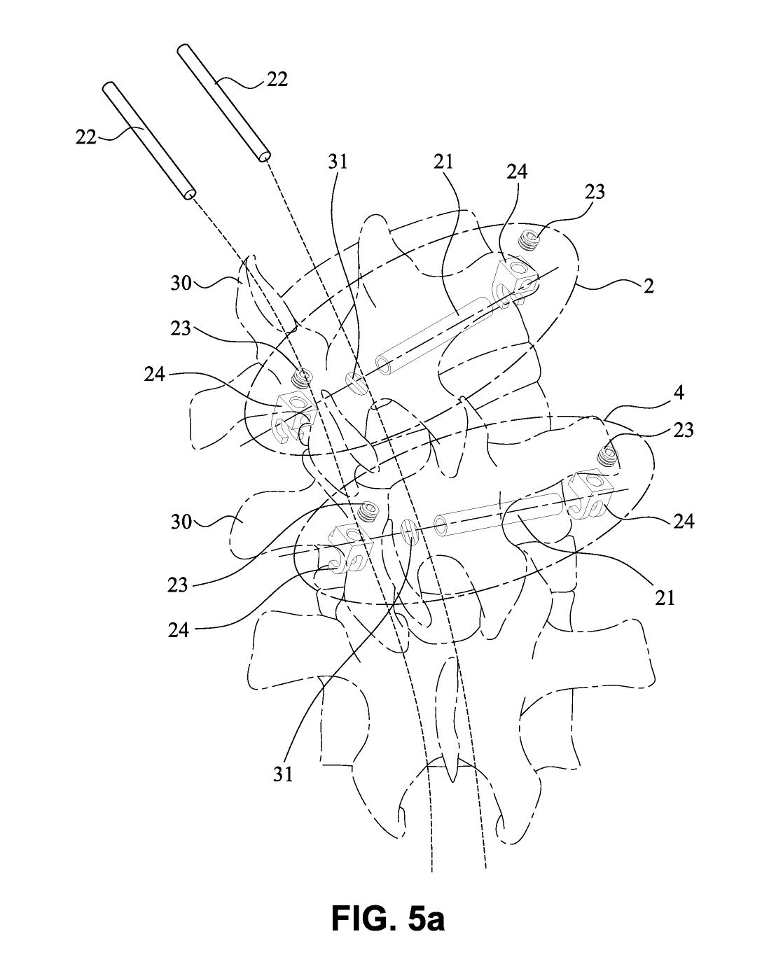

The fixing element 2 includes a transverse rod 21, two vertical rods 22, two screws 23 and two joints 24 (as shown in FIG. 4).

The transverse rod 21 is to provide securing effect to the vertebral body 30 transversely and may be formed to be a rod or an arcuate rod according to different practical requirements. The vertical rod 22 is a round cylinder, which provides vertical support to the vertebral body 30. The joints 24 has connecting holes defined in side faces respectively vertical to one another for extension of the transverse rod 21, the vertical rod 22 and the screw 23 respectively. Each joint 24 has a through hole 241, a vertical hole 242 defined to communicate with the through hole 241 and a cutout 243 communicating with both the vertical hole 242 and the through hole 241. It is to be noted that the vertical hole 242 is defined in a top portion of the joint 24 for extension of one end of the vertical rod 22 and the cutout 243 is defined in a bottom portion of the joint 24 for extension of one end of the transverse rod 21. The through hole 241 is defined to receive therein the screw 23 to secure the vertical rod 22 in the vertical hole 242 as well as the transverse rod 21 in the cutout 243. As the vertical rod 22 is much longer in length than that of the transverse rod 21, a different set of joint(s) may be added to the vertical rod 22 by extending a distal end of the vertical rod 22 through the vertical hole 242 of another joint 24. With such an arrangement, the user may apply the fixing element 2 to provide longitudinal as well as latitudinal support. In addition, the joint 24 has at least one extension 244 integrally extended from the body of the joint 24 for securing with the spine.

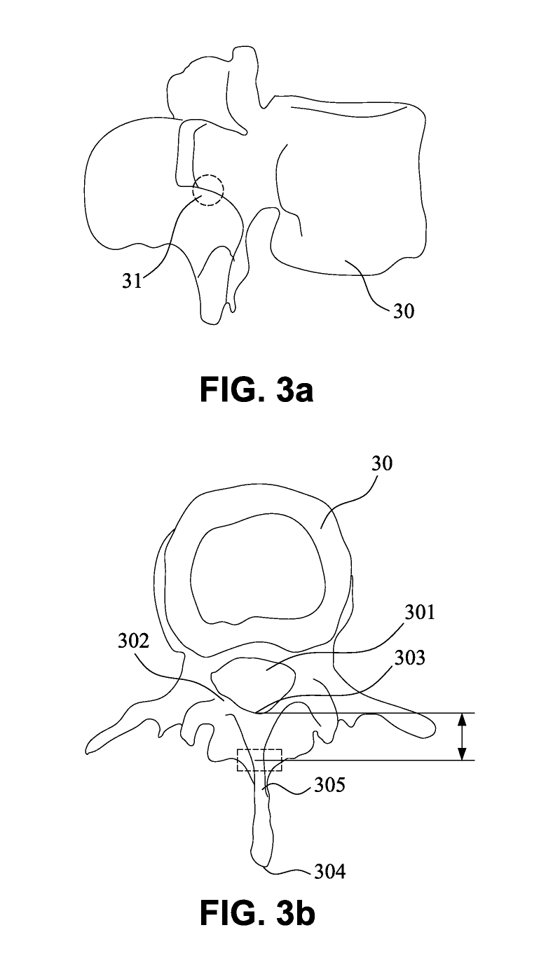

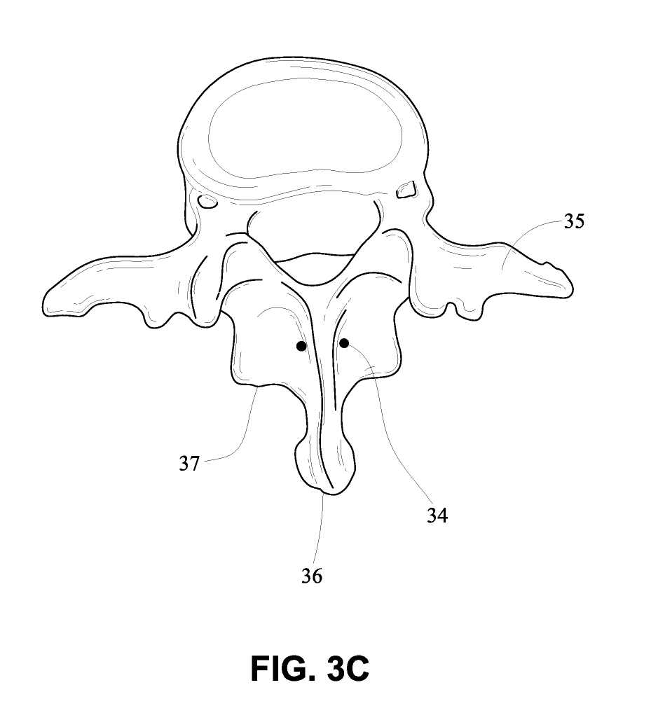

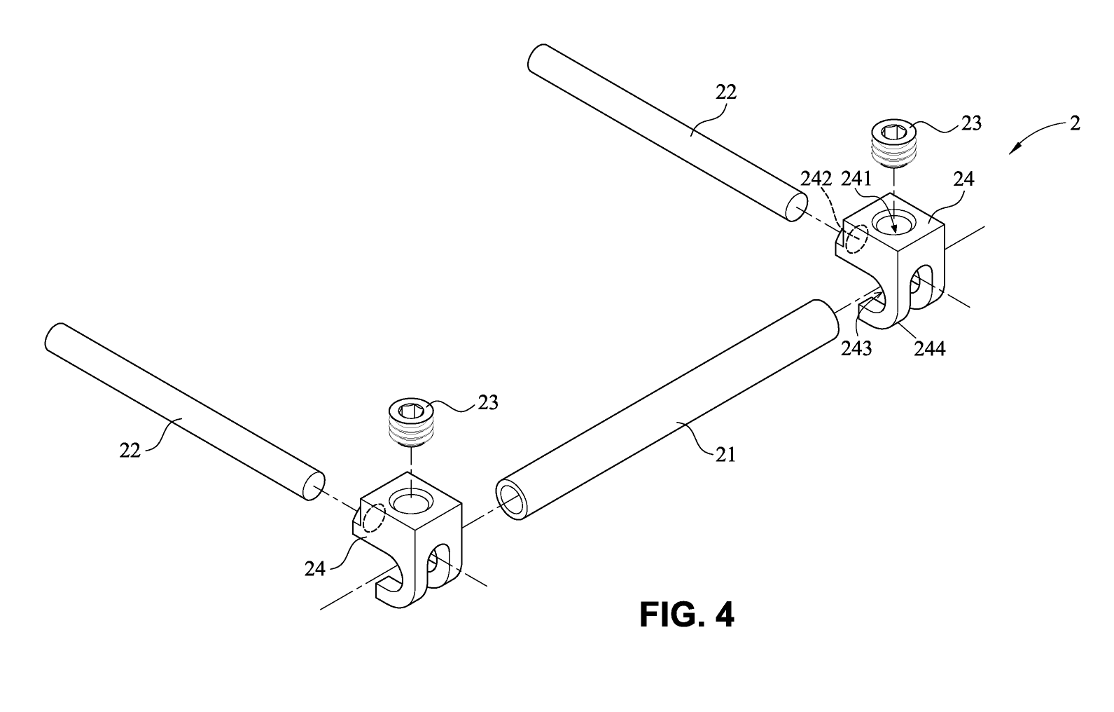

With reference to FIGS. 3a and 3b, the spine is constructed to have a plurality of vertebral bodies 30. A transverse hole 31 (as shown in FIG. 3a) of the vertebral body 30 is in an area between the bottom 303 of a lamina of vertebral arch situated at a vertebral foramen 301 and a top 305 of a spinous process 304. The transverse hole 31 (as shown in FIG. 3a) of the vertebral body 30 is further situated 7˜15 mm from the bottom 303 to the top 305.

With reference to FIG. 3c, which is a perspective view showing the location of a transverse hole 34 of a vertebra. The transverse hole is defined between transverse process 35 and spinous process 36 of a vertebra, wherein the hole 34 is transversally formed at a position of 7˜15 mm from the bottom of a lamina of vertebral arch 37 toward the top vertex of a spinous process 36.

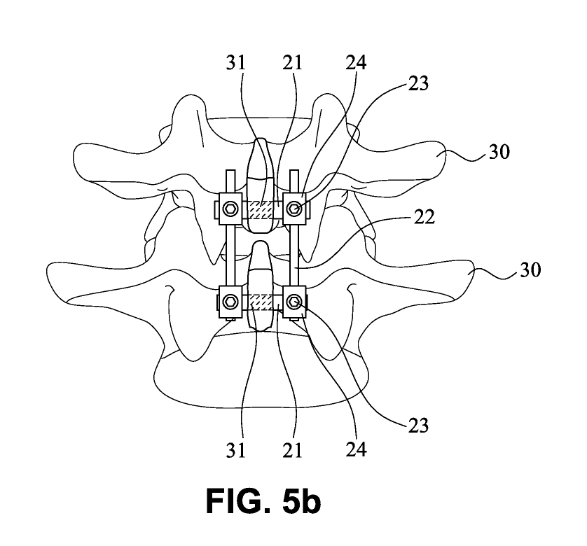

With reference to FIGS. 5a and 5b, which are applications of the fixation structure between the vertebral bodies 30. For instance, two sets of fixing elements 2 and 4 are respectively employed on the vertebral bodies 30 and connected to the joint 24 via the vertical rod 22, so that the vertebral bodies 30 can be securely fastened by the fixing element 2 and 4 to form a tic-tac-toe grid structure and that not only the horizontal but also the vertical directions of the vertebral bodies are supported and secured.

In order to combine more than two vertebral bodies 30 together, each fixing element 2 on each vertebral body 30 is connected to one another via the vertical rod 22, and then the transverse rod 21 and the vertical rod 22 are securely fastened by the screw 23 and the joint 24. In addition, the vertical rod 22 may be made to have different lengths to meet different requirements.

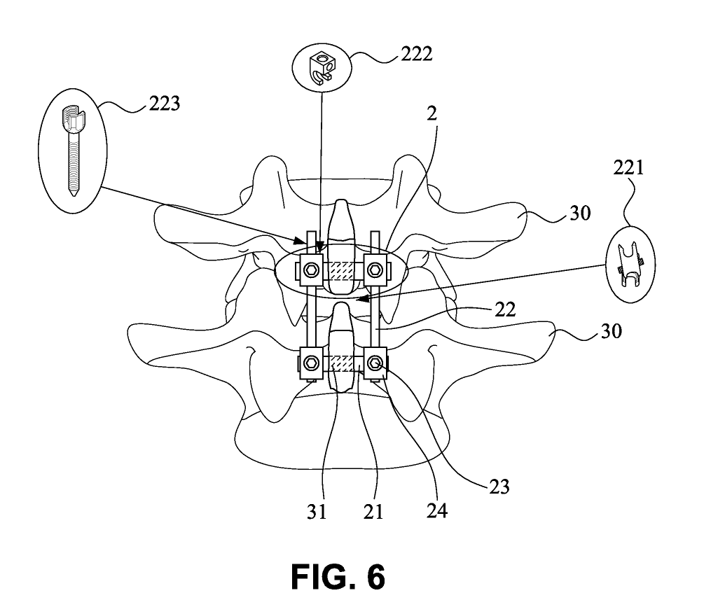

Also, the fixing element 2 mentioned above can also be used to connect the vertical rod 22 to other fixing devices (as shown in FIG. 6). For example, an interspinous process device 221 is set between the vertebral body 30 and the spinous process 304; a set of pedicle screw 222 is securely implanted on the two pedicles (not shown in the figure) of the vertebral body 30; or a set of spinal hook 223 with hook holding on two sides of the vertebral body 30 is provided as a result of this extended purpose. It is noted that the fixing element 2 can achieve other therapeutic purposes at the same time.

Therefore, the device for connecting a transverse beam at a triangular position of a vertebral lamina in accordance with the present invention is constructed by the fixing element 2. The transverse rod 21 is passed and situated between the bottom vertex 303 of the lamina of vertebral arch 302 and the top vertex 305 of a spinous process 304, and the two ends of the transverse rod 21 are respectively passed into the joint 24, and the joint 24 are both connected with the vertical rod 22 and the screw 23, wherein the transverse rod 21 and the vertical rod 22 are securely locked inside the joint 24 via the screw 23. In order to fix a plurality of vertebral body 30, each fixing element 2 is respectively fixed on each vertebral body 30 first, and connects with the other fixing element 2 of each vertebral body 30 via the vertical rods 21, so as to tightly fasten the spine in both vertical and horizontal orientations.

Many changes and modifications in the above described embodiment of the invention can, of course, be carried out without departing from the scope thereof. Accordingly, to promote the progress in science and the useful arts, the invention is disclosed and is intended to be limited only by the scope of the appended claims.

What is claimed is:

1. A fixing device adapted to extend through a hole defined between transverse process and spinous process of a vertebra, the fixing device comprising:
a transverse rod;
a vertical rod having a length longer than that of the transverse rod; and
a joint having a vertical hole to securely receive therein a portion of the vertical rod and a cutout communicating with the vertical rod to receive therein a portion of the transverse rod.

2. The fixing device as claimed in claim 1, wherein the hole is transversally formed at a position of 7˜15 mm from bottom of a lamina of vertebral arch toward top vertex of a spinous process.

3. The fixing device as claimed in claim 1, wherein the joint further has a through hole defined to communicate with the vertical hole and the cutout to receive therein a screw to secure the vertical rod in the vertical hole and the transverse rod in the cutout.

4. The fixing device as claimed in claim 1, wherein the vertical hole is defined in a top portion of the joint and the cutout is defined in a bottom portion of the joint.

5. The fixing device as claimed in claim 3, wherein the vertical hole is defined in a top portion of the joint and the cutout is defined in a bottom portion of the joint.
